(19) United States
(12) Patent Application Publication
(10) Pub. No.: US 2005/0107704 A1
(43) Pub. Date: May 19, 2005

(54) Motion analysis methods and systems for medical diagnostic ultrasound

(76) Inventors: Patrick L. Von Behren, Bellevue, WA (US); Jian-Feng Chen, Issaquah, WA (US)

Correspondence Address: Brinkds Hofer Gilson & Lione;Suite 3600, 455 N. City Front Plazat Drive, Chicago, IL, 60126, US

(21) Appl. No.: 10/713,453

(22) Filed: Nov. 14, 2003

(51) Int. Cl. A61B 8/00

(52) U.S. Cl. 600/443

(57) ABSTRACT

Medical imaging uses cyclical motion analysis. Phase and/or amplitude analysis of variation for spatial locations in a sequence of images over one or more heart cycles is performed. For phase analysis, selected phase information is cyclically isolated as a function of the heart cycle. For example, a sequence of three images is associated with three different times during the heart cycle. In one image, phases over one range are highlighted. In subsequent images, phases over different ranges are highlighted. By showing the sequence of images in a loop with the shifting phase throughout the sequence, wall contractions are easily visualized. For amplitude analysis, information associated with a selected frequency band, such as the constant and fundamental frequency bands, are isolated. Images are then generated in response to the isolated information. The images have reduced speckle content due to the lack of higher order frequency information. Some higher order frequency information may be allowed to remain or added to avoid motion blurring. The isolated information also more likely has well defined borders or edges as compared to the information with the full bandwidth.

### BACKGROUND

The present invention relates to phase and amplitude analysis. Imaging as a function of intensity variation is provided.

Intrinsic patient involuntary movements may cause motion of tissue and blood detectable in ultrasound images. For example, breathing, cardiac pulsations, arterial pulsations and muscle spasms are imaged. In the cardio vascular system, blood, cardiac and vessel movements determine normal and abnormal clinical states. For medical diagnostic ultrasound imaging, Doppler tissue imaging, strain rate imaging, M-mode imaging, examination of a sequence of B-mode images or detecting the outline or borders of chambers of a heart following wall motion are used to diagnose cardiac motion. Cardiac wall movement, valve movement and blood flow vary as a function of the heartbeat. The heart rate may be used in conjunction with imaging for visual assessment of cardiac motion. The visual assessment identifies abnormal operation and wall thickening. For muscular skeletal examinations,joint and ligament motions may provide diagnostic information.

In nuclear cardiology, gated blood pool studies are assembled from ECG gated two-dimensional images of the beating heart. The images are acquired by injecting radioactive substances and detecting gamma radiation from the body. A resulting sequence of images forms a representation of the heart during a composite cardiac cycle. The images are viewed in a CINE loop to assess cardiac wall motion. Since the heartbeat is periodic, a motion analysis may be performed using a Fourier analysis of detected tissue over the cardiac cycle for each image pixel. Two parametric images, one for the phase and one for the amplitude, indicate quantitative cardiac wall motion information. However, due to safety considerations, the level of radioactivity and resultant detector count rate are low for nuclear cardiology. Each image pixel is responsive to detected information over a time period of minutes. Temporal and spatial resolution may be limited by this count rate to no more than thirty images per cardiac cycle acquired over a long period of time.

Phase analysis has been performed in cardiac studies using ultrasound imaging. The onset of contractions during normal and abnormal beats is identified by phase analysis images. The phase for any given spatial location within an image is used to modulate a color display in a cyclic rainbow scale where red corresponds to 0° degrees and blue corresponds to 180°. Different shades or blends of these colors are used to represent other phases. Amplitude images are used to quantify the degree of wall motion.

### BRIEF SUMMARY

The present invention is defined by the following claims, and nothing in this section should be taken as a limitation on those claims. By way of introduction, the preferred embodiments described below include methods and systems for medical imaging with motion analysis. Phase and/or amplitude analysis of variation for spatial locations in a sequence of images over one or more heart cycles is performed. For phase analysis, selected phase information is cyclically isolated as a function of the heart cycle. For example, a sequence of three images is associated with three different times during the heart cycle. In one image, phases over one range are highlighted. In subsequent images, phases over different ranges are highlighted. By showing the sequence of images in a loop with the shifting phase throughout the sequence, wall contractions are easily visualized. For amplitude analysis, information associated with a selected frequency band, such as the constant and fundamental frequency bands, are isolated. Images are then generated in response to the isolated information. The images have reduced speckle content due to the lack of higher order frequency information. Some higher order frequency information may be allowed to remain or added to avoid motion blurring. The isolated information also more likely has well defined borders or edges as compared to the information with the full bandwidth.

In a first aspect, a method for medical imaging with motion analysis is provided. A phase of a cyclically varying imaging parameter is identified relative to a physiological cycle for each of a plurality of spatial locations in each of a plurality of image frames. A plurality of images corresponding to the plurality image frames is displayed. Each of the images is associated with a particular time segment within the physiological cycle. Spatial locations in one image associated with one phase are highlighted. Spatial locations in a subsequent image associated with a different phase are highlighted. The highlighting in each of the images is visually substantially the same.

In a second aspect, a method for ultrasound imaging with motion analysis is provided. A phase of a cyclically varying image parameter relative to the heart cycle is identified for a plurality of spatial locations in a sequence of image frames. Pixels in a sequence of images responsive to the image frames are highlighted. The highlighting shifts between images of the sequence as a function of a shifting phase interval.

In a third aspect, a method for ultrasound data processing with motion analysis is provided. Ultrasound data for each of a plurality of spatial locations is acquired over a physiological cycle. A sinusoidal waveform is matched with the ultrasound data for each of the spatial locations. Information associated with one frequency band is isolated from information associated with a different frequency band as a function of the matched sinusoid.

Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for motion analysis;

FIG. 2 is a flowchart diagram representing phase and amplitude analysis in one embodiment;

FIGS. 3a through 3c are graphic representations of images associated with various times during a heart cycle showing isolated phase information in one embodiment;

FIG. 4 is a graphical representation of one embodiment of matching a sinusoid to variation of an imaging parameter over a time period; and

FIGS. 5 and 6 are graphic representations of sinusoids fitted to an image brightness at two separate spatial locations varying over a heart cycle in one embodiment.

### DETAILED DESCRIPTION OF THE DRAWINGS AND PRESENTLY PREFERRED EMBODIMENTS

Phase and/or amplitude analysis of detected data is extended for ultrasound diagnostic imaging to provide additional motion information. For example, phase analysis is extended to provide a series of images with isolated phase information. The phase intervals for the isolated phase information shift as a function of time within the cardiac cycle. As a result, the contractions of the heart are visually highlighted in a same way but for different phases throughout the heart cycle. As another example, amplitude analysis is used to generate images with reduced speckle content or to better detect borders.

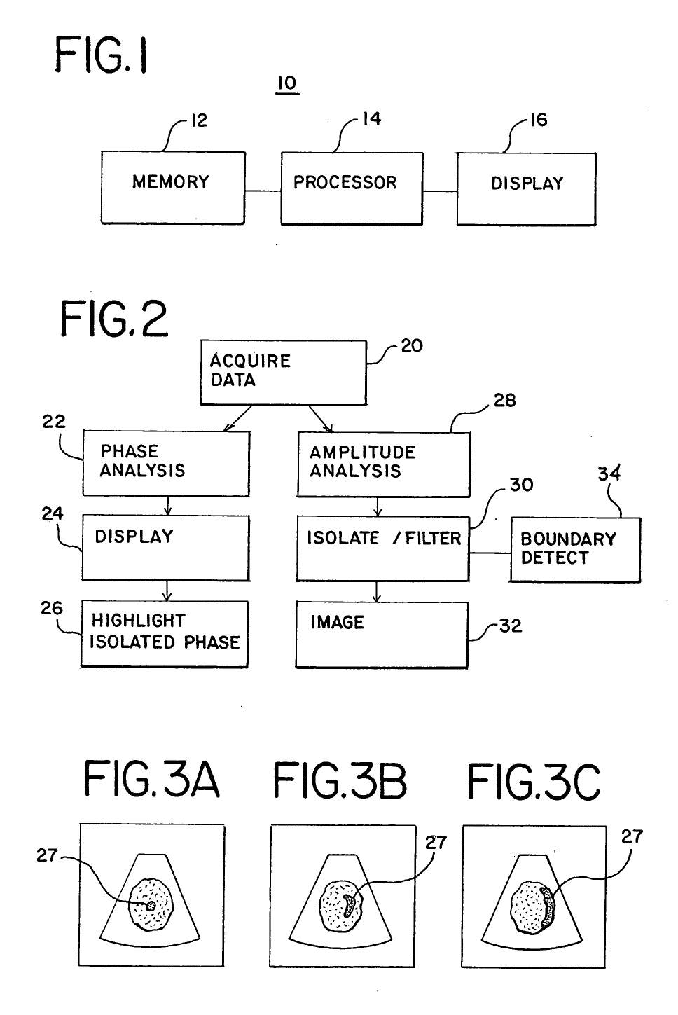

FIG. 1 shows a system 10 of one embodiment for phase and/or amplitude analysis. The system includes a memory 12, a processor 14 and a display 16. Additional, different or fewer components may be provided. For example, the system 10 is a medical diagnostic imaging system and includes a transmit beamformer, a transducer, a receive beamformer and a detector. As another example, the system 10 is a workstation or personnel computer. In alternative embodiments, the system 10 is a magnetic resonance imaging system or a computed tomography system.

The memory 12 is a RAM, hard drive, optical storage device, removable storage device, or other now known or later developed memory device. The memory 12 stores data formatted as a plurality of frames. Each of the plurality of frames is associated with a one, two or three dimensional region of the patient at a particular time or time period. In one embodiment, the memory 12 is formatted as a CINE loop memory for generating a sequence of ultrasound, MRI or CT images as a function of time.

The processor 14 is a general processor, application specific integrated circuit, digital signal processor, control processor, a processor used for data processing, an analog component, a digital component, combinations thereof or other now known or a later developed processing device. In one embodiment, a plurality of components are provided, such as an application specific integrated circuit for performing Fourier and inverse Fourier transforms and a separate processor for analyzing the transformed data. The processor 14 is operable to match a waveform, such as a sinusoid, to variations of an imaging parameter over a time period and determine phase and amplitude characteristics from the matched waveform. The processor 14 is also operable to use the phase and/or amplitude information for generating an image, an overlay or a portion of images.

The display 16 is a CRT, flat panel, plasma, LCD, projector, or other now known or a later developed display device. In one embodiment, the display 16 is a color display device, but black and white displays may be used. The display 16 receives image information directly from the processor 14 or from the processor 14 via one or more other components, such as a scan converter.

FIG. 2 shows a flow chart of one embodiment of a method for performing both phase and amplitude analysis. In alternative embodiments, only phase analysis or only amplitude analysis is performed. Additional, different or fewer acts may be associated with either of the phase or amplitude analysis. The method provides motion analysis, such as analysis of cardiac motion. For example, the phase analysis is used to highlight movement of a mechanical heart contraction wave during a heart cycle.

In act 20 data is acquired for each of a plurality of spatial locations. In one embodiment, the data acquired is ultrasound data. For example, B-mode or intensity data is acquired for a two dimensional region of a patient. Contrast agent, Doppler, M-mode or other data may be used in alternative embodiments. In yet other alternative embodiments, magnetic resonance imaging, nuclear medicine imaging, computed tomography, or another medical imaging modality data are acquired. The acquired data represents one, two or a three dimensional region of a patient. The data representing the region at any given time is formatted as a frame of data. Each frame of data includes one or more values for each of a plurality of spatial locations. Multiple frames are acquired over a physiological cycle. For example, thirty or more frames are acquired to represent different times within a physiological cycle. For any given spatial location, values are provided as a function of time through the multiple frames of data.

The signal-to-noise ratio of the acquired data may be improved by combining data from multiple physiological cycles. For example, an ECG trigger, analysis of the ultrasound data or other technique is used to identify a temporal location of each frame of data relative to the physiological cycle. Frames of data representing a same time within multiple physiological cycles are averaged. A weighted average or other combination may be used in alternative embodiments. Frames of data representing various times during a physiological cycle are interleaved together for a greater temporal resolution. Alternatively, frames of data representing similar but different times during the physiological cycle are combined. As a result, data from multiple physiological cycles are combined to represent a single composite physiological cycle. Data from multiple cycles may be combined to represent data for a lesser number of multiple cycles. In yet other alternative embodiments, frames of data are acquired over a single physiological cycle and used without further combination.

In act 22, a phase analysis is performed. A phase of a cyclically varying image parameter is identified relative to the physiological cycle. The phase is determined for each of a plurality of spatial locations. The spatial locations are associated with a single pixel in one embodiment, but may be associated with an average or other combination of each group of pixels. For example, an average intensity as a function of time is used for 7×7 or 15×15 regions of pixels. In one embodiment, a phase analysis is performed for multiple locations in a subset, a region of interest or for all the data within the plurality of frames of data.

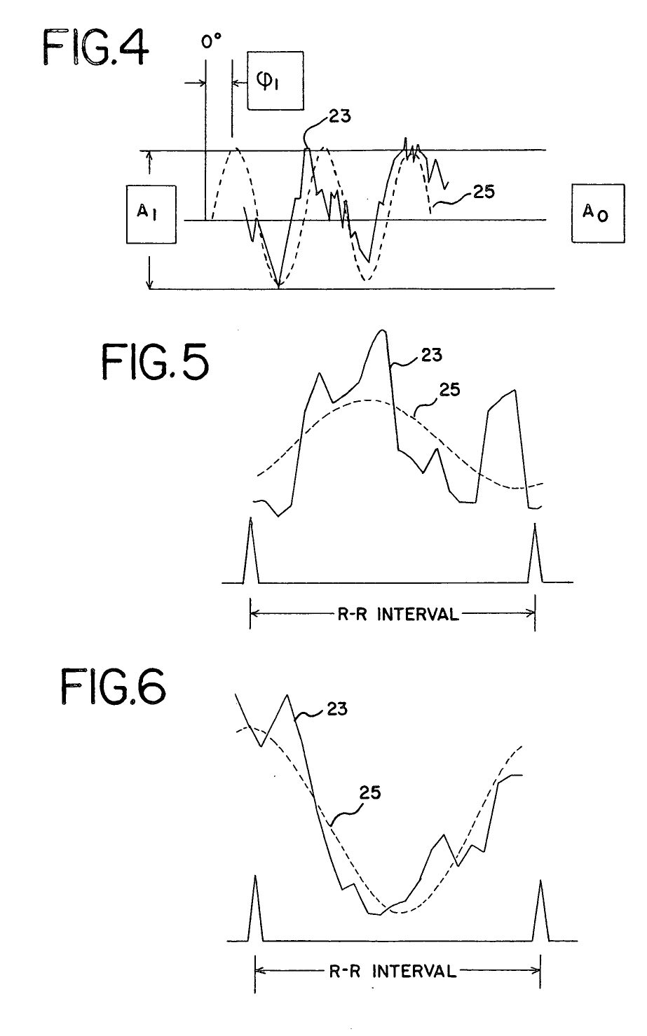

A sinusoid or sine wave is matched to variation in the B-mode values or other data during the physiological cycle. For example, FIGS. 4-6 show variation in a B-mode value 23 as a function of time. FIGS. 5 and 6 show B-mode values at two different spatial locations during a same heart cycle or R-wave to R-waved interval. The R-wave, heart cycle or other interval is determined using the ultrasound data or an ECG device. FIG. 4 shows the B-mode value 23 varying as a function of time over about two heart cycles. The B-mode variation as a function of time is represented as a time intensity curve, I(t). The brightness of the intensity varies cyclically as a function of time during a heart beat. The time intensity curve is mathematically represented by a Fourier series as:

$$I ( t ) = {\sum }_{K = 0 , n} {A}_{K} {ⅇ}^{- ⅈ ( k ω t - {φ}_{k} )} \qquad ( 1 )$$

where Aₖ is the amplitude of the k^{th} harmonic frequency, φₖ is the phase angle of the k^{th} harmonic frequency, ω is equal to 27 times the imaging or angular frequency, and n is the highest harmonic analyzed. The angular frequency ω is also equal to 2π divided by the period τ. τ is approximately equal to the time period of the heart cycle. The sinusoid includes one cycle for each heart cycle, and may include multiple cycles for higher harmonics. The heart cycle is determined using ECG or analysis of ultrasound data. Where frames of data are composited from multiple different heart cycles to represent a single cycle, the angular frequency or period corresponds to a composite cardiac cycle or averaged period.

Any of various processes are used to match the sinusoid to the time intensity curve for each spatial location. For example, Fourier, least squares fit, Hadamaard, wavelet, Walsh or other now known or a developed transforms are used to identify the desired or principal phase and amplitude components. Where frames of data representing only a portion of a heart cycle are provided, a least squares fit is used.

For one Fourier transform embodiment, a Fast Fourier Transform is used. The fundamental (i.e., first harmonic) is calculated in the frequency domain. Information associated with higher order harmonics is cancelled by identifying just the first harmonic. A principle amplitude of the fundamental frequency, a phase and an average or unchanging component (i.e., average amplitude) remain. The fast Fourier transform is performed for each of the spatial locations. The resulting fundamental and other desired components are inverse transformed to provide the sinusoid, such as the sinusoids 25 shown in FIGS. 4-6. In alternative embodiments, frequency components at higher harmonics up to the Nyquist sampling frequency are reduced but not eliminated or are selectively eliminated and reduced and may be used to analyze the motion.

The matched sinusoid 25 includes the DC or average value of the time intensity curve, the amplitude of the fundamental or other selected frequency and the phase angle associated with the selected frequency. These three parameters are provided as a function of time over a portion or the entire heart cycle for each spatial location. As a result of the match, the time intensity curve over the heart cycle is mathematically represented as:
|₁(t)=A₀+A₁ cos(ω·t−φ₁) (2)
where A₀ is the average value of the time intensity curve over a heart cycle or composite cycle, A₁ is the amplitude of the selected, such as fundamental, frequency, and φ₁ is the phase angle of the selected, such as fundamental, frequency. The sinusoid 25 provides isolated time intensity information used for imaging over a sequence of images.

The phase, represented as φ₁ in FIG. 4, is determined relative to the heart cycle. For example, the phase for the represented spatial location represented by FIG. 5 at the beginning of the heart cycle or at the R wave of the heart cycle is about 270 degrees. For the example of FIG. 6, the phase of the represented spatial location at the beginning of the heart cycle is at about 90 degrees. The phases at any other portions of the heart cycle are determined in a similar manner. For FIG. 4, the phase angle is approximately −16 degrees. While FIG. 4 represents determining a sinusoid 25 at the fundamental frequency, sinusoids associated with second or higher order harmonic may be determined. For any given image associated with a particular time within the heart cycle, the phase for each of the spatial locations is determined as a function of the matched sinusoid.

In act 24, a plurality of images is displayed. Each of the images is associated with a specific time interval within the physiological cycle and corresponds to the plurality of image frames used for performing the phase analysis. The displayed images include phase information. For example, a sequence of two-dimensional images is generated with at least one component of one or more pixels modulated as a function of the phase information. Anatomical reference information may be provided by superimposing the phase information on a background of the average or DC component. The average value of the pixels over the cardiac cycle is displayed in each of the images of the cardiac cycle. Since the average value is different for different pixels or spatial locations, an anatomical reference results. In one embodiment, a sequence of images is generated as B-mode images with the gray scale further modulated as a function of phase. Alternatively, the color or color characteristic is modulated as a function of phase. Alternatively, only phase information is used for generating the image. For example, a color or gray scale is modulated as a function of the amplitude for each of the spatial locations. In one embodiment, two-dimensional images are generated, but one- or three-dimensional images may be generated in other embodiments.

For some spatial locations, the time intensity curve may show little or no fundamental frequency variation over a heart cycle. For example, spatial locations associated with noise may result in low amplitude, random phase information. A threshold is applied in one embodiment, such as an amplitude threshold applied prior to trying to match a sinusoid, to avoid calculations associated with noise.

In act 26, isolated phase information is highlighted throughout a sequence of images. The highlighting shifts between images of the sequence as a function of a shifting phase interval. For example, spatial locations in one image associated with one phase or phase interval are highlighted. Spatial locations in a second image representing a different phase or phase interval are highlighted. The same or substantially same highlighting is used in each of the two sequential images, but for different spatial locations or for spatial locations associated with different phasing. The same, different or some of the same and some different spatial locations are highlighted in each subsequent image. The highlighting is visually the same to show a contraction across the sequence of images of over time.

In one embodiment, pixels or spatial locations associated with a phase or phase interval for highlighting are darkened. For example, gray scale values or color phase values are set to a darker color or gray scale. In one embodiment, spatial locations associated with the desired phase or phase interval are set to black. Black highlighting of the pixels having a zero degree phase represents the onset of contraction. As an example, 30 frames of data and associated images are generated with a frame rate of 33 milliseconds per frame. The heart beat or heart cycle is about one second long. Accordingly, each frame is associated with a phase angle range of about 12 degrees if equally divided (i.e. 360 degrees representing the heart cycle divided by 30 frames). For the first image, spatial locations associated with zero to 11 degrees of phase are highlighted. For the second or subsequent image, spatial locations associated with 12 to 23 degrees are highlighted. The process repeats until the 30th frame where spatial locations associated with 348 degrees to 359 degrees are highlighted. In alternative embodiments, the phase ranges associated with highlighting in each image overlaps with a phase range of another image. The phase ranges between the images are adjacent for each immediately subsequent image, but one or more phase angles may be skipped or repeated across multiple images.

For any given image, spatial locations associated with one range are highlighted and spatial locations associated with another range of phase angles are free of highlighting or have different highlighting. The spatial locations for the same or similar highlighting vary as a function of time within the heart cycle as the phase angle or phase angle range shifts throughout the heart cycle. As a result, motion of the heart or the mechanical contraction wave is shown through isolation of phase information throughout the sequence. The mechanical wave mimics the electrical activation sequence. As a result, the contraction of different regions at different times within the heart cycle is viewed. Motion associated with an abnormally moving portion of the heart may be more easily identified. For example, irregular motion is identified for electrophysiology ablation procedures. By using isolated phase information to show motion within a sequence of images over a heart cycle, the sick portion of the heart is identified for removal or ablation with radiofrequency electrodes.

In one embodiment, noise is removed by temporally filtering the original time domain image sequence and/or in the phase domain. For example, a window of two or more frames is averaged across the sequence of images. The temporal averaging includes the highlighting in one embodiment, but may be performed prior to highlighting in other embodiments. By temporally filtering with the highlighted information, a smoother transition between frames is provided.

For contrast agent imaging or other imaging to identify a specific portion of the heart, a mask is used to hide or remove phase information for spatial locations outside of the desired region. For example, the amplitude of the DC component of the matched sinusoid, the average B-mode intensity, the maximum B-mode intensity, the amplitude of the fundamental component or other value is applied to a threshold to identify regions of interest. As an alternative to an amplitude threshold, the masking is performed by a manual trace by the user, automatic detection of a boundary or other process. For contrast agent imaging, areas within the cardiac chambers are likely to have higher amplitudes. As a result of masking, images may be less complicated and more focused on a region of interest. In alternative embodiments, B-mode values or other information is displayed outside a region of interest while the phase information or a combination of phase and other information are displayed for regions of interest.

Isolation of phase information as a function of time within the cardiac cycle may be used to enhance pace maker assessment procedures. The wall motion is examined throughout a sequence of contractions. Using an ECG or other pace maker feedback, the sequence of images is aligned relative to the pacemaker trigger. For a phase angle of zero, the beginning of the pacemaker trigger is provided. For example, highlighting is provided by adding a shade of red. At the beginning of a pacemaker trigger, a spot associated with the pacemaker electrode is shown as red. As the sequence of images continues a wave of red moves outward from the spot.

The isolated phase information is used in other embodiments for other diagnosis. FIGS. 3A-3C show a sequence of three images at different portions of the heart cycle. In FIG. 3A, pixels associated with one range of phases are highlighted as represented by the dark region 27. In FIG. 3B, the dark region 27 expands and moves to the right. In FIG. 3C, the dark region expands further and moves further to the right. The dark regions in FIG. 3B and 3C are associated with different sequential ranges of phase. Since the same darker region is used in each of the images, the highlighting is visually the same and shows movement of the contraction across the images.

Referring again to FIG. 2, an amplitude analysis is performed in act 28 in an additional or alternative embodiment. The amplitude analysis is performed for determining a characteristic through data processing or for generating an image. The amplitude information is determined as discussed above by matching a sinusoidal waveform with the data, such as ultrasound B-mode time intensity data for each of a plurality of spatial locations. For example, the data is transformed to a frequency domain by a Fast Fourier transform as discussed above. The data in the frequency domain is then used to isolate particular information and an inverse Fourier transform is performed.

In act 30, information associated with one frequency band is isolated from information associated with a different frequency band. The isolation is performed for each spatial location of interest. For example, information associated with the fundamental frequency band and the unvarying or average amplitude component are isolated from information associated with higher order harmonics by fitting the sinusoidal to the time intensity curve for a given spatial location. The best match sinusoid 25 provides a fundamental amplitude, A₁ and the average amplitude A₀ as shown in FIG. 4. In alternative embodiments, a sinusoid representing a best match second harmonic or other higher order harmonic is determined. By matching the sinusoidal waveform to the time intensity curve, information associated with undesired frequencies is effectively set to zero or removed. The best matching, such as calculated through a Fourier transform, operates to low-pass filter the time intensity curve. The low-pass filtering reduces or eliminates speckle.

In act 32, B-mode or intensity images are generated using the sinusoidal waveform 25 as a function of time within the heart cycle. A different amplitude along the sinusoid is selected as a function of time. Where the spatial locations represent a two-dimensional region, each spatial location within the region is associated with an intensity selected from the sinusoidal waveform 25. Spatial filtering may reduce region based transitions. Images are generated with the intensities as a function of time. Three-dimensional images may also be generated as a function of time from the sinusoidal waveform 25.

Due to the reduction in higher order information, motion blurring may result. Additional terms or fractions of terms from the Fourier series, such as fractions of higher order information, are added back to the sinusoidal waveform 25 to reduce motion blurring. The information is added in either the frequency domain or the spatial domain. Information is added to the transform data in the frequency domain or to the inverse transform data in the spatial domain. For example, in the frequency domain, the second harmonic Fourier term is added to the sinusoidal waveform 25 of the first harmonic or fundamental frequency. Less speckle reduction may be provided, but motion representation may be improved. Rather than calculating the sinusoidal waveform of the fundamental frequency alone, the fundamental and second harmonic are determined in the frequency domain using the transform provided by:

$$I ( t ) = {\sum }_{l = 0 , 2} {A}_{l} {ⅇ}^{- l ( ω t - {φ}_{l} )} \qquad ( 3 )$$

Different or additional harmonic terms may be added in the frequency domain, such as the third or fractional harmonics.

In the spatial domain, information from the different frequency bands is added to the inverse transformed ultrasound data (e.g., the matched sinusoidal waveform 25). For example, the originally acquired B-mode information is added to the intensity information determined by the amplitude analysis. In one embodiment, an infinite impulse response (IIR) filter is used to combine the information. The amplitude analyzed speckle reduced information is weighted with one value (e.g., α) and the original image information is weighted with another value (e.g., 1-α). The relative weights are adjusted as a function of the desired amount of speckle reduction and associated motion blurring. The relative weights are either precalculated, calculated as a function of feedback or manually set.

In act 34, the isolated information, such as represented by the sinusoidal waveform 25, is used to detect a boundary or segment of one type of data from another type of data. While shown as using amplitude information, parametric images from the average non-varying amplitude component, the various harmonic amplitude components or the various phase components may be used to detect distinct boundaries in alternative embodiments. Any of various edge detection processes may be used, such as applying an edge enhancement operator or filter (e.g. Laplacian). Other gradient-based, amplitude threshold or now known or later developed edge detection techniques may be used. The edge detection is applied in the spatial domain using the parametric images from the matched sinusoids 25 (i.e. the intensities filtered by phase or amplitude analysis). In alternative embodiments, edge detection is applied in the frequency domain. Different combinations of analysis may be used. For example, a phase image is used to isolate a region of interest and amplitude images are used for refining the border detection within the region of interest. Due to the reduction in speckle information and isolating fundamental frequency band information, borders may be more accurately detected for cardiac diagnosis.

While the invention has been described above by reference to various embodiments, it should be understood that many changes and modifications can be made without departing from the scope of the invention. It is therefore intended that the foregoing detailed description be regarded as illustrative rather than limiting and that it be understood that it is the following claims, including all equivalents, that are intended to define the spirit and the scope of the invention.

1. A method for medical imaging with motion analysis, the method comprising:
(a) identifying a phase of a cyclically varying imaging parameter relative to a physiological cycle for each of a plurality of spatial locations in each of a plurality of image frames;
(b) displaying a plurality of images corresponding to the plurality of image frames, each of the plurality of images associated with a different time within the physiological cycle;
(c) highlighting spatial locations in a first image of the plurality of images associated with a first phase; and
(d) highlighting spatial locations in a second image of the plurality of images associated with a second phase, the second phase different than the first phase and the second image corresponding to the different time than the first image;
wherein the highlighting of (c) is visually substantially the same highlighting of (d) at one of the same spatial locations, different spatial locations and combinations thereof.

2. The method of claim 1 wherein (a) comprises, for each of the plurality of spatial locations:
(a1) matching a sinusoid to variation in B-mode values during the physiological cycle; and
(a2) identifying the phase of the sinusoid relative to the time within the physiological cycle for each of the plurality of image frames

3. The method of claim 2 wherein (a1) comprises performing a Fourier transform and (a2) comprises identifying the phase as a phase angle at a fundamental frequency from data responsive to (a1).

4. The method of claim 1 wherein (a) comprises identifying the phase for spatial locations comprising single pixels.

5. The method of claim 1 wherein (b) comprises generating B-mode images.

6. The method of claim 1 wherein (c) and (d) comprise setting the imaging parameter to a darker shade for spatial locations associated with the first phase and second phase, respectively.

7. The method of claim 1 wherein (c) comprises highlighting spatial locations associated with the first phase being a first range of phases and (d) comprises highlighting spatial locations associated with the second phase being a second range of phases, the second range being free of overlap with the first range.

8. The method of claim 7 wherein the first range of phases ends where the second range of phases begins, the second image being immediately subsequent to the first image.

9. The method of claim 1 further comprising:
(e) highlighting images subsequent to the first and second images, the spatial locations being highlighted in different images being associated with different phases.

10. The method of claim 1 wherein (b), (c) and (d) comprises highlighting movement of a mechanical heart contraction wave during the physiological cycle being a heart cycle.

11. The method of claim 1 wherein (c) comprises highlighting associated with the first phase and free of highlighting associated with the second phase and (d) comprises highlighting associated with the second phase and free of highlighting associated with the first phase.

12. The method of claim 1 further comprising:
(e) combining frames of data from multiple of the physiological cycles, the combined frames of data representing a single physiological cycle and being the plurality of image frames.

13. The method of claim 1 wherein (b) comprises generating three-dimensional images.

14. The method of claim 1 further comprising:
(e) synchronizing with a pace maker.

15. The method of claim 1 wherein (c) and (d) comprises showing motion associated with a sick portion of a heart.

16. A method for ultrasound imaging with motion analysis, the method comprising:
(a) identifying a phase of a cyclically varying imaging parameter relative to a heart cycle for each of a plurality of spatial locations in each of a plurality of image frames; and
(b) highlighting pixels in a sequence of images responsive to the plurality of image frames, the highlighting shifting between images of the sequence as a function of a shifting phase interval.

17. A method for ultrasound data processing with motion analysis, the method comprising:
(a) acquiring ultrasound data for each of a plurality of spatial locations over a physiological cycle;
(b) matching a sinusoid waveform with the ultrasound data for each of the pluralities of spatial locations;
(c) isolating information associated at least one frequency band from information associated with a different frequency band for each of the plurality of spatial locations as a function of the matched sinusoid; and
(d) adding information from the different frequency band to the isolated information.

18. The method of claim 17 wherein (b) comprises performing a fast Fourier transform.

19. The method of claim 17 wherein (a) comprises acquiring the data over a plurality of heart cycles and combining the data to represent a single heart cycle.

20. The method of claim 17 wherein (c) comprises isolating information associated with an unvarying component and a fundamental frequency component by reducing values for information associated with second harmonics of the fundamental frequency component.

21. The method of claim 17 wherein (c) comprises isolating information associated with a harmonic of a higher order than a fundamental frequency component by reducing values for information associated with at least the fundamental frequency component.

22. The method of claim 17 wherein (a) comprises acquiring data representing contrast agents.

23. The method of claim 17 further comprising:
(e) generating images of intensities as a function of time responsive to (d).

24. The method of claim 23 wherein (e) comprises generating three-dimensional images.

25. The method of claim 17 wherein (d) comprises adding the information from the different frequency band to the isolated information in the frequency domain.

26. The method of claim 17 wherein (d) comprises adding the information from the different frequency band to the isolated information in the spatial domain.

27. The method of claim 17 wherein (b) comprises:
(b1) transforming the ultrasound data for each of the plurality of spatial locations into a frequency domain;
(b2) isolating information associated with at least one frequency band from information associated with a different frequency band for each of the plurality of spatial locations; and
(b3) inverse transforming the isolated information.

28. A method for ultrasound data processing with motion analysis, the method comprising:
(a) acquiring ultrasound data for each of a plurality of spatial locations over a physiological cycle;
(b) matching a sinusoid waveform with the ultrasound data for each of the pluralities of spatial locations; and
(c) isolating information associated at least one frequency band from information associated with a different frequency band for each of the plurality of spatial locations as a function of the matched sinusoid.
(d) detecting a boundary from data responsive to (c).

29. The method of claim 28 wherein (b) comprises:
(b1) transforming the ultrasound data for each of the plurality of spatial locations into a frequency domain;
(b2) isolating information associated with at least one frequency band from information associated with a different frequency band for each of the plurality of spatial locations; and
(b3) inverse transforming the isolated information.

30. The method of claim 28 wherein (d) comprises detecting the boundary from amplitude data.

31. The method of claim 28 wherein (d) comprises detecting the boundary from phase data.
